(19) United States
(12) Patent Application Publication
(10) Pub. No.: US 2025/0024938 A1
(43) Pub. Date: Jan. 23, 2025

(54) TOOTHBRUSH SANITIZING DEVICE

(71) Applicant: SMILE MAKERS, LLC, San Diego, CA (US)

(72) Inventor: Kami Hoss, San Diego, CA (US)

(21) Appl. No.: 18/775,980

(22) Filed: Jul. 17, 2024

Related U.S. Application Data
(60) Provisional application No. 63/528,296, filed on Jul. 21, 2023.

(51) Int. Cl. A46B 17/06; A46B 13/02; A46B 15/00; A61C 17/22; A61L 2/10; A61L 2/24; A61L 2/26; H02J 50/10

(52) U.S. Cl. CPC A46B 17/065; A46B 13/02; A46B 15/0085; A61C 17/224; A61L 2/10; A61L 2/24; A61L 2/26; H02J 50/10; A61L 2202/11; A61L 2202/121; A61L 2202/122; A61L 2202/14; A61L 2202/16

(57) ABSTRACT

A toothbrush sanitizing device can include a drying element, a sanitizing element, and a charging element. The toothbrush sanitizing device can at least partially enclose a toothbrush and sanitize and charge the toothbrush when the toothbrush is stored inside the toothbrush sanitizing device. The toothbrush sanitizing device can include a removable top that can function as a rinsing cup. The toothbrush sanitizing device can also include a removable bottom that can be open to conveniently clean out accumulated waste.

### INCORPORATION BY REFERENCE TO ANY PRIORITY APPLICATIONS

This application claims priority to U.S. Provisional Application No. 63/528,296, filed Jul. 21, 2023, and which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The field relates to toothbrush sanitizing devices, and, in particular, to sanitizing device that can sanitize, store and charge an electric toothbrush.

### Description of the Related Art

Although toothbrushes and other oral care devices have been used by humans for centuries, proper maintenance and sanitization of toothbrushes remain a problem for many people. Many users do not effectively sanitize their toothbrushes. Moreover, sanitizing toothbrushes may be inconvenient, especially for electric toothbrushes. Accordingly, there remains a continuing need for improved toothbrush sanitizing devices.

### SUMMARY

An aspect of this disclose is a toothbrush sanitizing device for an electric toothbrush. The device comprises a main body having a chamber to receive the electric toothbrush, a bottom cover removably attached to a bottom side of the main body, an upper cover removably attached to a top side of the main body, a charging coil disposed on an interior surface of the main body and configured to charge the electric toothbrush, one or more ultraviolet lights configured to at least partially clean the electric toothbrush, a heater or other drying device configured to remove at least some moisture from the electric toothbrush, and a sensor configured to identify a presence of the electric toothbrush in the toothbrush sanitizing device.

A variation of the aspect above further comprises a magnetic coupler disposed on the interior surface of the main body and configured to removably couple the electric toothbrush to the toothbrush sanitizing device.

A variation of the aspect above is, wherein, when the electric toothbrush is inside the chamber of the main body, the electric toothbrush is positioned upside down such that a brush head of the electric toothbrush is closer to the bottom side of the main body than the top side of the main body.

A variation of the aspect above is, wherein, when the electric toothbrush is inside the chamber of the main body, the electric toothbrush is positioned right side up such that a brush head of the electric toothbrush is closer to the top side of the main body than the bottom side of the main body.

A variation of the aspect above is, wherein the bottom cover comprises vents.

A variation of the aspect above is, wherein the upper cover comprises a rinsing cup when removed from the main body.

A variation of the aspect above is, wherein the upper cover is transparent to visible light.

A variation of the aspect above further comprises a switch disposed on an exterior surface of the main body and configured to indicate an operational status of the toothbrush sanitizing device.

A variation of the aspect above is, wherein the charging coil is disposed proximate a handle of the electric toothbrush when the electric toothbrush is disposed in the chamber of the main body.

A variation of the aspect above is, wherein the charging coil is disposed proximate a base of the electric toothbrush when the electric toothbrush is disposed in the chamber of the main body.

A variation of the aspect above is, wherein the induction sensor is a Hall sensor.

A variation of the aspect above further comprises a printed circuit board (PCB) disposed proximate a brush head of the electric toothbrush.

A variation of the aspect above is, wherein the one or more ultraviolet lights are disposed on the PCB.

A variation of the aspect above is, wherein the heater comprises a heating resistor disposed on the PCB.

A variation of the aspect above is, wherein the heater comprises a fan or a blower positioned within the chamber of the main body in addition to the heater.

A variation of the aspect above is, wherein the heater comprises a fan or a blower positioned within the chamber of the main body in the alternative to the heater.

Another aspect is direct to a toothbrush sanitizing device for an electric toothbrush. The device comprises a main body having a chamber to receive the electric toothbrush, a bottom cover removably attached to a bottom side of the main body, an upper cover removably attached to a top side of the main body, one or more ultraviolet lights configured to at least partially clean the electric toothbrush, a heater configured to remove at least some moisture from the electric toothbrush, and a magnetic coupler disposed on an interior surface of the main body and configured to removably couple the electric toothbrush to the toothbrush sanitizing device.

A variation of the aspect above further comprises a charging coil disposed on the interior surface of the main body and configured to inductively charge the electric toothbrush.

A variation of the aspect above further comprises a sensor configured to identify a presence of the electric toothbrush in the toothbrush sanitizing device.

A variation of the aspect above is, wherein, when the electric toothbrush is inside the chamber of the main body, the electric toothbrush is positioned upside down such that a brush head of the electric toothbrush is closer to the bottom cover than the upper cover.

A variation of the aspect above is, wherein, when the electric toothbrush is inside the chamber of the main body, the electric toothbrush is positioned right side up such that a brush head of the electric toothbrush is closer to the top cover than the bottom cover.

Another aspect is directed to a method of storing and sanitizing an electric toothbrush. The method comprises inserting the electric toothbrush into a chamber of a main body of a toothbrush sanitizing device, removably attaching an upper cover to a top side of the main body of the toothbrush sanitizing device, detecting a presence of the electric toothbrush inside the main body of the toothbrush sanitizing device with a sensor, and activating a plurality of functions of the toothbrush sanitizing device, The plurality of functions comprises inductively charging the electric toothbrush with a charging coil disposed on an interior surface of the main body of the toothbrush sanitizing device, at least partially sanitizing the electric toothbrush with one or more ultraviolet lights, and removing at least some moisture from the electric toothbrush with a heater or other drying device.

A variation of the aspect above is, wherein inserting the electric toothbrush into the chamber of the main body of the toothbrush sanitizing device comprises inserting the electric toothbrush upside down such that a brush head of the electric toothbrush is closer to the bottom side of the main body of the toothbrush sanitizing device than the top side of the main body of the toothbrush sanitizing device.

A variation of the aspect above is, wherein inserting the electric toothbrush into the chamber of the main body of the toothbrush sanitizing device comprises inserting the electric toothbrush right side up such that a brush head of the electric toothbrush is closer to the top side of the main body of the toothbrush sanitizing device than the bottom side of the main body of the toothbrush sanitizing device.

A variation of the aspect above further comprises removably coupling the electric toothbrush to the toothbrush sanitizing device with a magnetic coupler disposed on the interior surface of the main body.

A variation of the aspect above further comprises removing a bottom cover to at least partially clean a bottom side of the main body of the toothbrush sanitizing device.

Another aspect is directed to a toothbrush. The toothbrush comprises a brush head, a handle having a bottom side and a top side, wherein the brush head is connected to the handle at the top side of the handle, a charging unit disposed inside the handle, the charging unit having a charging circuitry, the charging circuitry configured to inductively gain power from a charging coil of a toothbrush sanitizing device and charge a battery of the toothbrush, a magnet disposed in the handle and configured to couple to a coupler on the toothbrush sanitizing device, and a status indicator disposed on the handle and positioned closer to the bottom side than the top side of the handle.

A variation of the aspect above further comprises a portable charging box configured to magnetically attach to the handle of the toothbrush via the magnet and provide power to the toothbrush.

Another aspect is directed to a toothbrush. The toothbrush comprises a brush head, a handle having a bottom side and a top side, wherein the brush head is connected to the handle at the top side of the handle, a charging unit on the handle, the charging unit having charging circuitry, the charging circuitry configured to inductively gain power from a charging coil of a toothbrush sanitizing device and charge a battery of the toothbrush, a magnet disposed in the handle and configured to couple to a coupler on the toothbrush sanitizing device.

A variation of the aspect above is, wherein the charging unit is disposed proximate the bottom side of the handle.

A variation of the aspect above is, wherein the charging circuitry comprises a charging coil.

A variation of the aspect above further comprises a status indicator disposed on the handle.

Another aspect is direct to a charger for a toothbrush. The charger comprises a body having a surface sized and shaped to conform to a portion of an exterior surface of the toothbrush, a charging circuitry inside the body configured to charge a battery of the toothbrush, a magnet in the body configured to removably attach the body to the toothbrush.

### BRIEF DESCRIPTION OF THE DRAWINGS

The present disclosure is described with reference to the accompanying drawings, in which like reference characters reference like elements, and wherein:

FIG. 1A is a front, bottom, right perspective view of an embodiment of a toothbrush sanitizing device disclosed herein.

FIG. 1B is a front view of the toothbrush sanitizing device in FIG. 1A.

FIG. 1C is a rear view of the toothbrush sanitizing device in FIG. 1A.

FIG. 2A is a rear perspective view of an exemplary electric toothbrush.

FIG. 2B is a rear perspective view of a charger configured to charge the electric toothbrush in FIG. 2A.

FIG. 2C shows the charger of FIG. 2B attached to the electric toothbrush of FIG. 2A.

FIG. 3A is a front cross-sectional view of another embodiment of a toothbrush sanitizing device disclosed herein.

FIG. 3B is a left side cross-sectional view of the toothbrush sanitizing device in FIG. 3A, taken along line 3B-3B.

FIG. 3C is a rear cross-sectional view of the toothbrush sanitizing device in FIG. 3A.

FIG. 3D is a rear cross-sectional view of another embodiment of a toothbrush sanitizing device disclosed herein, having a fan.

FIG. 4 shows a front cross-sectional view of the toothbrush sanitizing device from FIG. 3A and a rear view of the electric toothbrush in FIG. 2, illustrating how the electric toothbrush can be positioned inside the toothbrush sanitizing device.

FIG. 5A shows a toothbrush sanitizing device of FIG. 3A with a top cover removed, and the electric toothbrush of FIG. 2 inserted.

FIG. 5B is a front side of a PCB on the toothbrush sanitizing device of FIG. 5A, facing a chamber of the toothbrush sanitizing device.

FIG. 5C is a back side of the PCB of FIG. 5B, opposite the front side.

FIG. 5D is a charging control panel on the toothbrush sanitizing device of FIG. 5A.

FIG. 6 illustrates a child's toothbrush sanitizing device used with a child's electric toothbrush.

### DETAILED DESCRIPTION

Various embodiments disclosed herein relates to a toothbrush sanitizing device, e.g., a device for sanitizing an electric toothbrush. The toothbrush sanitizing device may include one or more electrical elements to enable various functionality including, for example, charging the toothbrush, drying the toothbrush, and sanitizing the toothbrush. More specifically, the toothbrush sanitizing device can also be configured to house and protect the toothbrush between uses while cleaning and charging the toothbrush. In some embodiments, the toothbrush sanitizing device can include a removable bottom cover attached to a bottom side of the toothbrush sanitizing device. The removable bottom cover can be configured to allow removal of accumulated waste and cleaning of the bottom side of the toothbrush sanitizing device. In some embodiments, the removable bottom cover can further include vents for air flow and dripping water from the toothbrush. In some embodiments, the toothbrush sanitizing device can also include a removable top cover attached to a top side of the toothbrush sanitizing device. The removable top cover can be used as a rinsing cup when removed. In some embodiments, the removable top cover can also be transparent or semi-transparent to work with an electric toothbrush inside the toothbrush sanitizing device that include lights to function as a night light.

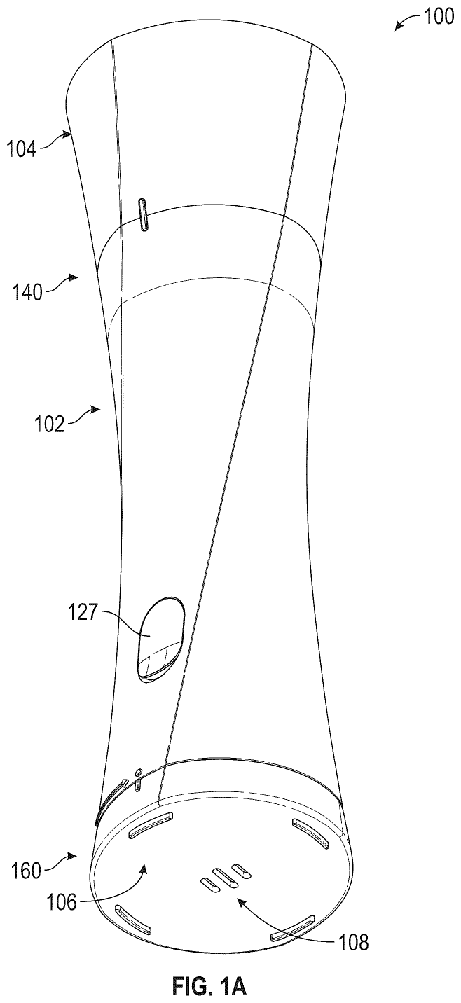

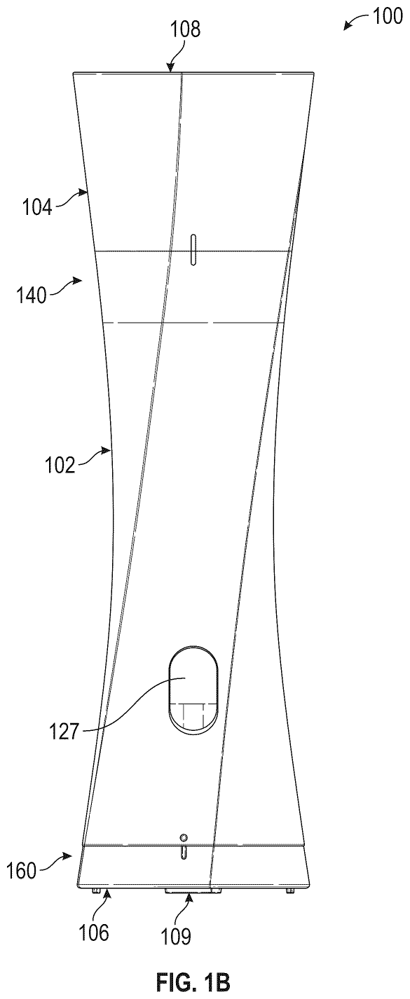

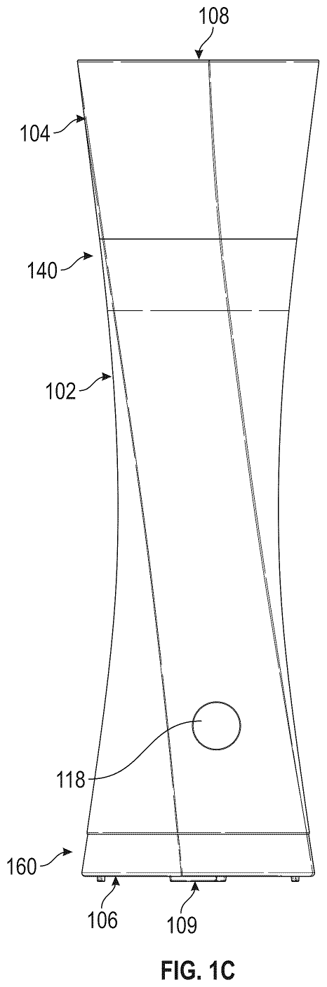

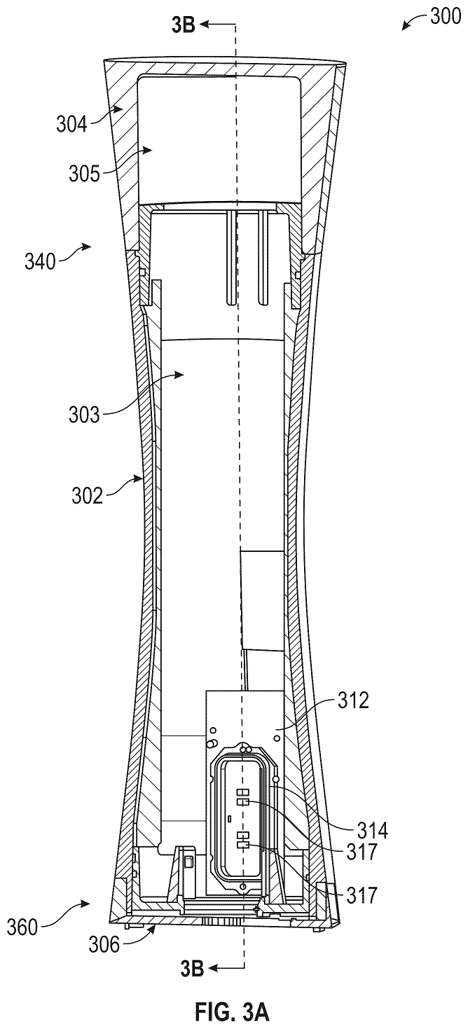

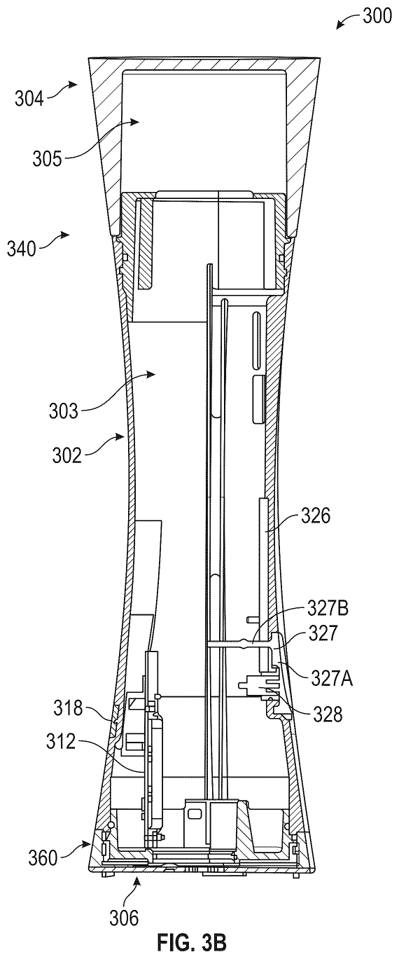

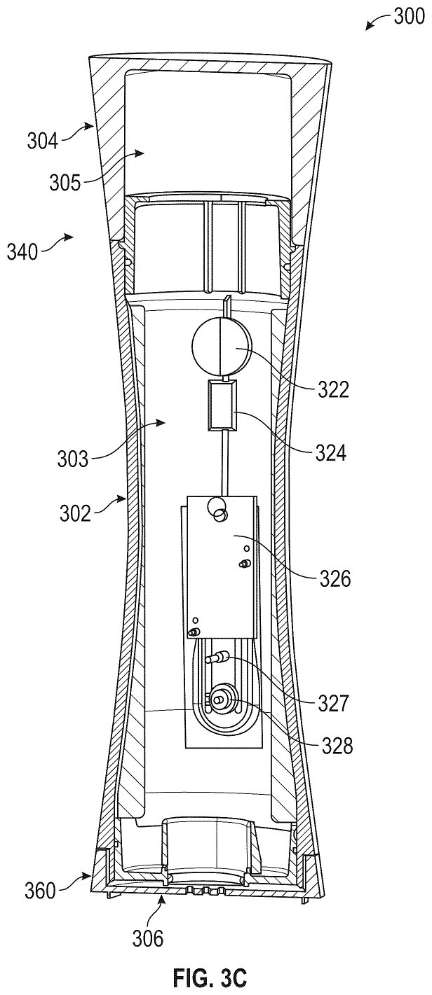

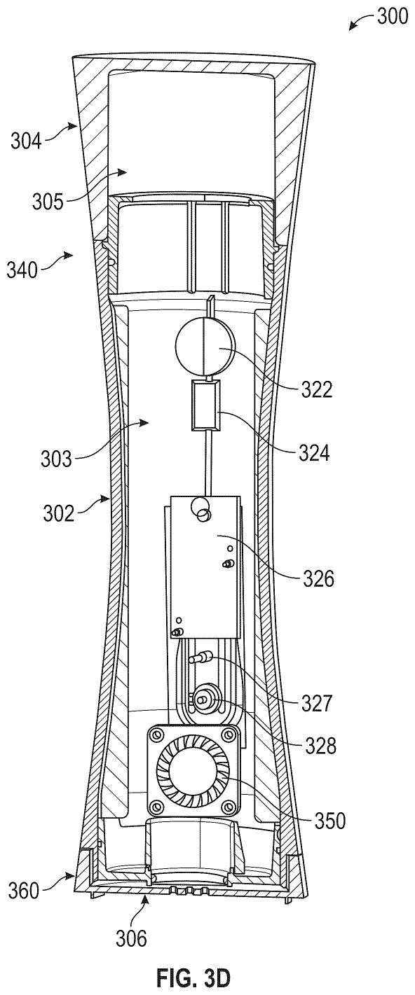

In various embodiments, an exemplary embodiment of a toothbrush sanitizing device 100 can include a main body 102, a top cover 104, and a bottom cover 106, forming an at least partially enclosed body. In some embodiments, the toothbrush sanitizing device 100 can have an elongated body that is widest at the top, narrowest at an intermediate waist portion, and at an intermediate width at the bottom as shown in FIGS. 1A-IC. In some embodiments, a chamber (see FIG. 3A-3C) of the main body 102 can at least partially enclose a toothbrush. In other embodiments, the chamber (see FIG. 3A-3C) of the main body 102 may completely enclose a toothbrush.

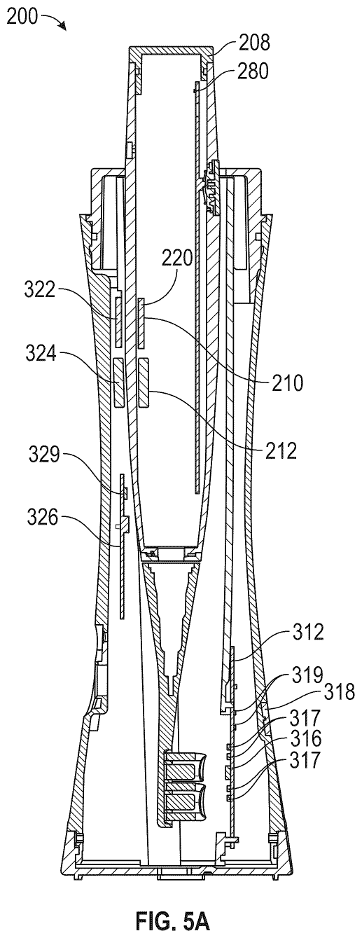

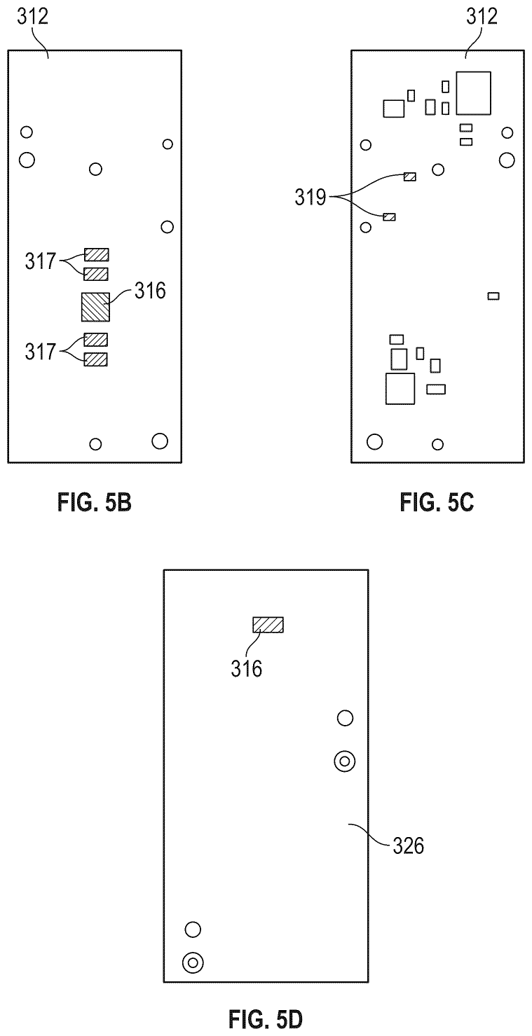

In some embodiments, the main body 102 of the toothbrush sanitizing device 100 can include a top cover 104 removably attached to a top side 140 of the main body 102 via, e.g., a threaded connection, a snap-fit connection, a bayonet mount, gravity, etc. in some embodiments, the top cover 104 may be configured to sit on the top side 140 of the main body 102 without a fastening means. In some embodiments, the top cover 104 may be transparent, partially transparent, or semi-transparent to visible light such that an interior space of the toothbrush sanitizing device 100 can be visible from outside the device 100. It may be desirable to have a top cover 104 that is at least partially transparent so that, for example, a status indicator on the toothbrush, which is inside the toothbrush sanitizing device 100, can be monitored from outside the device 100. The status indicator on the toothbrush can be an indicator that shows whether the toothbrush is being charged or has been fully charged. In some embodiments, the status indicator can also be configured to indicate a status of the sanitizing, for example, the status indicator can show a first color when sanitizing is underway, and switches to a second color when sanitizing is finished. In some embodiments, as shown in FIG. 5A, the status indicator can include a LED light 280 that can be included on an electric toothbrush 200 and visible from a transparent or semi-transparent base 208 at a bottom end of the electric toothbrush 200. In some embodiments, the LED light 280 can show a certain color once the electric toothbrush 200 is fully charged to indicate the charging state, and also act as a night light, visible through the top cover 104 of the toothbrush sanitizing device 100. In some embodiments, for example, the LED light 280 can show and maintain a red color when the electric toothbrush 200 is fully charged as the color red does not affect melatonin level and sleep quality of a person. Having the top cover 104 that is at least partially transparent can allow a user to conveniently monitor the status of the toothbrush while the toothbrush is being covered and sanitized. In some embodiments, the top cover 104 may include a chamber (see FIG. 3A-3C) configured to hold a liquid (e.g., water, mouthwash, etc.) such that the top cover can be used as a rinsing cup when removed from the toothbrush sanitizing device 100. As shown in FIGS. 1B-C, the top cover 104 can have a top surface 108 that is substantially planar so that the top cover 104 can be self-standing and/or support the toothbrush sanitizing device 100 to stand on a planar surface.

In some embodiments, the toothbrush sanitizing device 100 can further include a bottom cover 106 removably attached to a bottom side 160 of the main body 102 via, e.g., a threaded connection, a snap connection, bayonet mount, etc. In some embodiments, the bottom cover 106 may be easily removable such that the bottom side 160 of the main body 102 can be cleaned by removing accumulated waste around the bottom side 160 of the main body 102 and at the bottom cover 106 of the toothbrush sanitizing device 100. In some embodiments, the bottom cover 106 can also include vents 108 (see FIG. 1A) to allow air flow and liquid escaping to reduce bacteria growth. As shown in FIGS. 1B-C, the bottom cover 106 can have a bottom surface 109 that is substantially planar so that the bottom cover 106 can be self-standing and/or support the toothbrush sanitizing device 100 to stand on a planar surface.

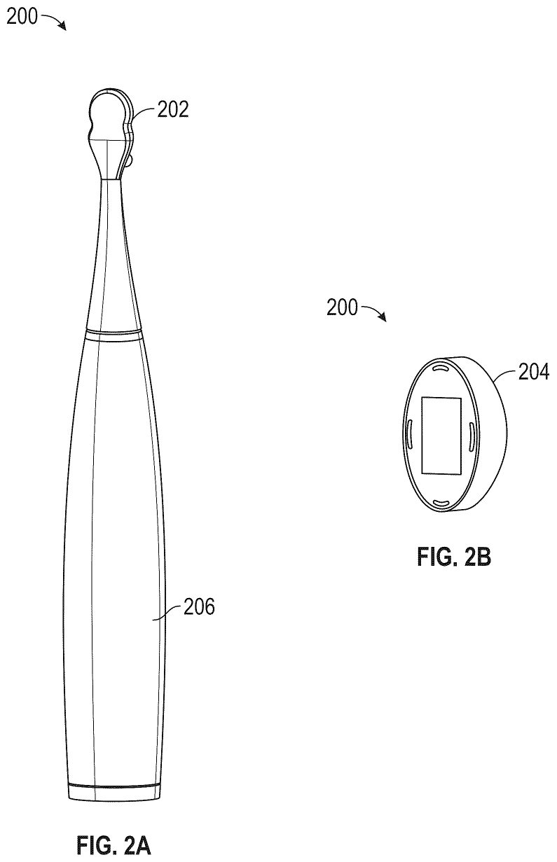

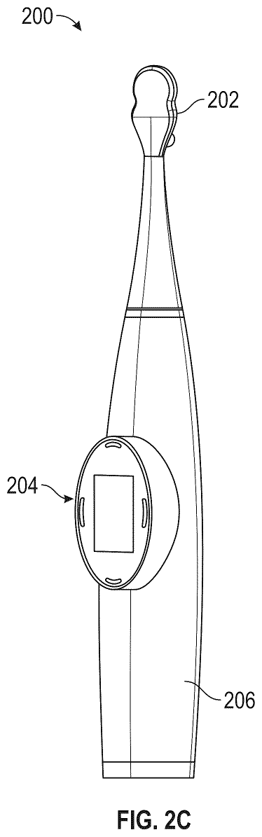

In various embodiments, a toothbrush sanitizing device disclosed herein can be used with different kinds of toothbrushes, including various manual toothbrushes and electric toothbrushes. In some embodiments, it may be desirable to use the toothbrush sanitizing device with an electric toothbrush to store, sanitize, as well as charge the toothbrush at the same time. For example, the toothbrush sanitizing device 100 can be used with an electric toothbrush 200 illustrated in FIG. 2A. The electric toothbrush 200 can include a brush head 202 attached to a handle 206. The handle 206 of the electric toothbrush 200 can include various mechanical and/or electrical parts, for example, a magnet 212 to couple the toothbrush 200 to a magnet coupler of a toothbrush sanitizing device disclosed herein (see FIG. 5A), when the electric toothbrush 200 is inserted into the toothbrush sanitizing device. As further described below, in some embodiments, the magnet 212 of the electric toothbrush 200 can also allow the toothbrush sanitizing device to detect presence of the electric toothbrush 200 inside the toothbrush sanitizing device by a magnetic field of the magnet 212. In some embodiments, the handle 206 of the electric toothbrush 200 can also include a charging unit having charging circuitry 210. In some embodiments, the charging circuitry 210 can include a charging coil in correspondence to a charging coil of the charging device on the toothbrush sanitizing device 100/300 to charge the electric toothbrush by induction (see FIG. 5A), when the electric toothbrush 200 is inserted into the toothbrush sanitizing device 100/300. As shown in FIG. 5A, the handle 206 of the electric toothbrush 200 can also include a battery 220 connected to the charging circuitry 210 to store power from the charging circuitry 210.

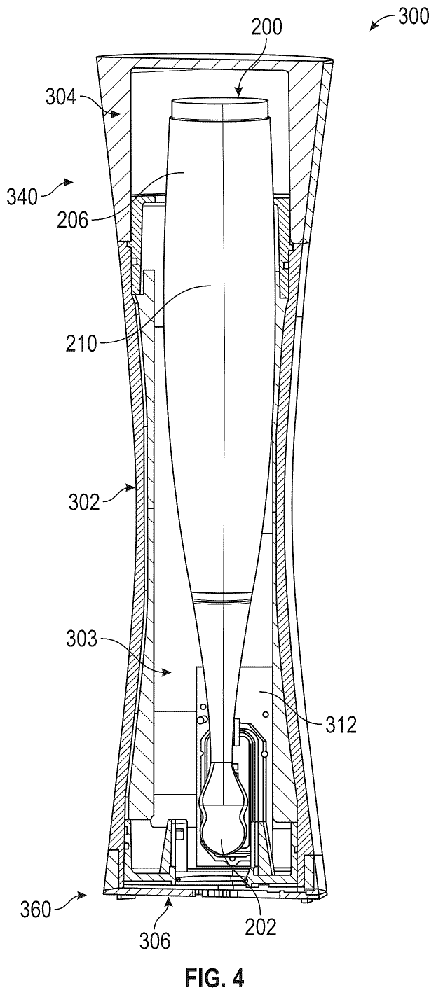

Advantageously, a toothbrush can be placed into the toothbrush sanitizing device 100 upside down, with a brush head of the toothbrush positioned closer to the bottom side 160 than to the top side 140 of the main body 102 of the toothbrush sanitizing device 100 (See FIGS. 4 and 5A). With the brush head of the toothbrush facing downward, residual fluid and waste on the brush head can drip down to a bottom side of the toothbrush sanitizing device 100 instead of flowing towards the handle of the toothbrush and contaminating the handle.

Another aspect of this disclosure includes a charging box 204 (see FIG. 2B) removably attachable to the handle 206 of the electric toothbrush 200. In some embodiments, the charging box 204 of the electric toothbrush 200 can include a charging unit configured to provide power to the electric toothbrush 200, in correspondence with the charging circuitry 210 of the electric toothbrush 200. The electric toothbrush 200 may store power in the battery 220 gained through the charging circuitry 210. In some embodiments, the charging box 204 can include a magnet such that the charging box 204 can be magnetically attached to the handle 206, and easily removed when not needed, for example, when the electric toothbrush 200 is inserted into the toothbrush sanitizing device 100/300. In some embodiments, the charging box 204 may be portable such that the charging box 204 freely movable, and attached to the electric toothbrush 200 when needed. In some embodiments, the charging box 204 may be secured to, e.g., a wall or a counter, such that the electric toothbrush 200 can be attached to the charging box 204 by magnet and on, e.g., the wall or the counter.

In various aspects, a toothbrush sanitizing device 300 disclosed herein can include various mechanical and electrical components for coupling a toothbrush and cleaning the toothbrush. As shown in FIG. 3A-3C, the toothbrush sanitizing device 300 can similarly include a main body 302 having a chamber 303 to receive the toothbrush, a top cover 304 removably attached to a top side 340 of the main body 302, and a bottom cover 306 removably attached to a bottom side 360 of the main body 302.

In some embodiments, at least some of these mechanical and electrical components can be positioned inside a chamber 303 of a main body 302 of the toothbrush sanitizing device 300. For example, in some embodiments, the toothbrush sanitizing device 300 can include a printed circuit board (“PCB”) 312 disposed inside the chamber 303 of the main body 302. In some embodiments, the PCB 312 can be positioned proximate a portion of the chamber 303 that may receive the brush head of the toothbrush (See FIGS. 3A-B). As shown in FIGS. 1C and 3B, in various embodiments, the toothbrush sanitizing device 100 or 300 can also include an switch 118 or 318 disposed on an exterior surface of the main body 102 or 302 and configured to switch between on and off or other operation modes of the toothbrush sanitizing device 100 or 300. With reference to FIG. 3B, the switch 318 can also be configured to electrically connect to the PCB 312 and can control one or more of electrical components on the PCB 312 (e.g., sanitizing lights, drying device, etc.). In some embodiments, the switch 318 can also act as a status indicator configured to indicate an operational status of the toothbrush sanitizing device 100 or 300. In some embodiments, as shown in FIGS. 5A and 5C, one or more LED lights 319 can be disposed on a back side of the PCB 312 and behind the switch 318, the one or more LED lights 319 configured to generate different light colors or patterns visible through the switch 318. In some embodiments, the one or more LED lights 319 can be configured to indicate an operational status of the toothbrush sanitizing device 300 (e.g., charging, drying, sanitizing, not sanitized, sanitized, etc.), for example, one or more LED lights 319 can display a first color when the toothbrush sanitizing device 300 is sanitizing the electric toothbrush 200, and switches to a second color when the toothbrush sanitizing device 300 has finished sanitizing the electric toothbrush 200. In some embodiments, the PCB 312 can include a controller mounted to the PCB 312. The PCB 312 can be connected to the switch 318 and be configured to send signals to the one or more of the electrical components on the PCB 312 to control the electrical components to turn on, turn off, turn on for a period of time, change between operational modes, etc.

In various embodiments, the toothbrush sanitizing device 300 can include one or more sanitizing lights 316 to sanitize the toothbrush. The one or more sanitizing lights 316 may be configured to at least partially clean the toothbrush, and in some embodiments, specifically the brush head of the toothbrush. In some embodiments, the one or more sanitizing lights 316 can be positioned on a front side of the PCB 312, proximate the bottom side 360 of the toothbrush sanitizing device 300 to target the brush head of the toothbrush as illustrated in FIGS. 3A, and 4-5B. In some embodiments, the one or more sanitizing lights 316 can comprise ultraviolet LED lights or any other lights capable of sanitizing a toothbrush.

In some embodiments, the toothbrush sanitizing device 300 can further include a drying device to remove at least some moisture from the toothbrush. In some embodiments, the drying device can include at least one of a heater, a blower and a fan. In some embodiments, the heater 317 of the toothbrush sanitizing device 300 can be a resistive heater, a heating film, or any other suitable drying device. In some embodiments, a heater 317 in the form of a resistive heater can be provided in or on the PCB 312, proximate the bottom side 360 of the toothbrush sanitizing device 300 to dry the toothbrush, including a brush head of the toothbrush (see FIGS. 5A and 5B). For example, in some embodiments, the resistive heater can be mounted to the PCB 312 by solder balls. In some embodiments, a silicone pad 314 can be disposed over the PCB 312 to separate and protect electrical components on the PCB 312 from moisture introduced by the toothbrush. Also FIGS. 5A-5B only show one sanitizing light 316, and multiple heaters 317, in other embodiments, there can be multiple sanitizing lights 316 and just one heater 317, multiple sanitizing lights 316 and multiple heaters 317, or just one sanitizing light 316 and one heater 317. As illustrated in FIG. 3D, the drying device can include, alternatively, or in addition to the heater, at least one fan 350 to facilitate air circulation within the chamber 303 and to dry the brush head of the toothbrush. Although the drying device in FIG. 3D is illustrated as a fan, the drying device could be one or more blowers or any other apparatus or instrument that would facilitate air circulation within the toothbrush sanitizing device 300 or would facilitate moisture evaporation within the toothbrush sanitizing device 300. In some embodiments the at least one fan 350 can be mounted proximate to a portion of the chamber 303 that may receive the brush head of the toothbrush. The at least one fan 350 may be mounted to the chamber 303 by at least one of screws, fasteners, adhesives, and coupling, for example. Alternatively, or in addition, the at least one fan 350 may be positioned in, on, or proximate the PCB 312, proximate the bottom side 360 of the toothbrush sanitizing device 300.

In various embodiments, the toothbrush sanitizing device 300 can further include a charging device 322 disposed inside the chamber 303 of the main body 302 as shown in FIG. 3C. The charging device 322 may be positioned proximate a handle of an electric toothbrush inserted in the toothbrush sanitizing device 300 to conveniently charge the electric toothbrush. In some embodiments, the charging device 322 can include a charging coil positioned proximate a portion of the chamber 303 that corresponds to a charging unit on the toothbrush (when the toothbrush is disposed inside the toothbrush sanitizing device 300) to charge by induction. As shown in FIG. 3C, in some embodiments, the charging device 322 including a charging coil can be connected to a charging control panel 326 and a charging female jack 328. The charging female jack 328 can be configured to connect to an external power source that provide power to the toothbrush sanitizing device 300. For example, as shown in FIG. 4, when the electric toothbrush 200 is placed inside the toothbrush sanitizing device 300, the charging device 322 may be positioned proximate the charging circuitry 210 on the electric toothbrush 200 such that the charging device 322 can provide power to the battery 220 of the electric toothbrush 200 through the charging circuitry 210.

As shown in FIGS. 1A-B and 3B-C, in various embodiments, the toothbrush sanitizing device 100 or 300 can also include a charging hole cover 127 or 327. In some embodiments, the charging hole cover 327 may include an external portion 327A configured to protect the charging female jack 328 from the external environment when inserted into the charging female jack 328, and an internal portion 327B configured to stop the charging hole cover 327 from fall out of the toothbrush sanitizing device 300 when the external portion 327A is removed from the charging female jack 328.

In other aspects, the toothbrush sanitizing device 300 can include a coupler 324 configured to removably couple the electric toothbrush inside the toothbrush sanitizing device 300. In some embodiments, the coupler can be a magnet positioned inside the chamber 303 of the main body 302, corresponding to a magnetic component on the electric toothbrush so that the toothbrush can be easily secured in a desirable position inside the toothbrush sanitizing device 300 as illustrated in FIG. 4.

Other aspects of the toothbrush sanitizing device 300 disclosed herein can include a sensor 329 configured to sense presence of an object inside the toothbrush sanitizing device 300 and activate one or more functions when an object is sensed. In some embodiments, the sensor 329 can be positioned on the charging control panel 326, proximate a handle or a magnet of an electric toothbrush inserted inside the toothbrush sanitizing device 300 (see FIGS. 5A and 5D). For example, in some embodiments, the sensor can be a Hall effect sensor configured to sense a magnetic field of the toothbrush. In some embodiments, the sensor can cause one or more of the heating, sanitizing, and drying functions to be activated when presence of at least a portion of a toothbrush is detected inside the toothbrush sanitizing device 300.

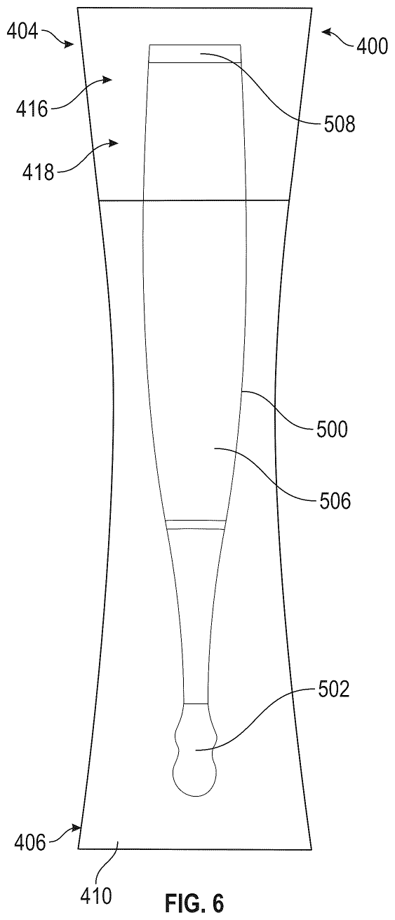

In yet another embodiment, another toothbrush sanitizing device 400 is disclosed. Unless otherwise noted, the toothbrush sanitizing device 400 can include similar elements that operates similarly as that of the toothbrush sanitizing device 100 or 300 disclosed above. As illustrated in FIG. 6, the toothbrush sanitizing device 400 can be used with another embodiment of an electric toothbrush 500, with the electric toothbrush 500 inserted right side up in the toothbrush sanitizing device 400. The electric toothbrush 500 may include a brush head 502 at a top end and a base 508 at a bottom end. In some embodiments, the base 508 of the electric toothbrush 500 may include a charging unit. Advantageously, including the charging unit at the base 508 of the electric toothbrush 500 instead of inside a handle 506 of the electric toothbrush 500 can allow the electric toothbrush 500 to have a smaller size (e.g., shorter and/or thinner) than the electric toothbrush 200 disclosed above, and therefore, suitable for use by a child. In some embodiments, the toothbrush sanitizing device 400 can also be configured to be smaller in size than the toothbrush sanitizing device 100/300 above, and suitable for use with a child's toothbrush.

In some embodiments, in correspondence with the charging unit positioned at the base 508 of the s electric toothbrush 500, the toothbrush sanitizing device 400 can include a charging unit 410 positioned at a bottom portion 406 of the toothbrush sanitizing device 400. In some embodiments, as the electric toothbrush 500 is positioned with the brush head 502 proximate a top portion 404 of the toothbrush sanitizing device 400, the toothbrush sanitizing device 400 may include one or more sanitizing lights 416 (e.g. ultraviolet LED lights) positioned at the top portion 404 of the toothbrush sanitizing device 400. In some embodiments, the one or more sanitizing lights 416 may be built into a removable top cover at the top portion 404 of the toothbrush sanitizing device 400. In some embodiments, the toothbrush sanitizing device 400 may also include a display configured to show a digital timer 418 at the top portion 404 of the toothbrush sanitizing device 400. In some embodiments, the digital timer 418 may be positioned on the removable top cover, and configured to start counting down when the one or more sanitizing lights 416 are activated.

In some embodiments, the electric toothbrush 500 may also be equipped with a gyroscope and/or an accelerometer configured to track brushing motion of a user. In some embodiments, the electric toothbrush 500 can also include Bluetooth connectivity to a software implemented on an external smart device and configured to receive data from the gyroscope and/or an accelerometer to track usage and duration, report to the users, parents of the users, and/or dentists, and display tutorials to teach users the correct brushing technique.

In accordance with various embodiments disclosed herein, the electric toothbrushes 200 and 500 may also be used without a toothbrush sanitizing device. The electric toothbrushes 200 and 500 may be used with other charging devices, such as the charging box 204 described above. In some embodiments, the electric toothbrushes 200 and 500 can be used with other charging devices, e.g., one that hangs on a wall, stands on a counter, etc.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Moreover, as used herein, when a first element is described as being “on” or “over” a second element, the first element may be directly on or over the second element, such that the first and second elements directly contact, or the first element may be indirectly on or over the second element such that one or more elements intervene between the first and second elements. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while features of the toothbrush sanitizing device are presented in a given arrangement, alternative embodiments may perform similar functionalities with different arrangements and/or geometries, and some features may be deleted, moved, added, subdivided, combined, and/or modified. Each of these features may be implemented in a variety of different ways. Any suitable combination of the elements and acts of the various embodiments described above can be combined to provide further embodiments. The accompanying claims and their equivalents are intended to cover such forms or modifications as would fall within the scope and spirit of the disclosure.

What is claimed is:

1. A toothbrush sanitizing device for an electric toothbrush, the device comprising:
a main body having a chamber to receive the electric toothbrush,
a bottom cover removably attached to a bottom side of the main body,
an upper cover removably attached to a top side of the main body,
a charging coil disposed on an interior surface of the main body and configured to charge the electric toothbrush,
one or more ultraviolet lights configured to at least partially clean the electric toothbrush,
a heater configured to remove at least some moisture from the electric toothbrush, and
a sensor configured to identify a presence of the electric toothbrush in the toothbrush sanitizing device.

2. The device of claim 1, further comprising a magnetic coupler disposed on the interior surface of the main body and configured to removably couple the electric toothbrush to the toothbrush sanitizing device.

3. The device of claim 1, wherein, when the electric toothbrush is inside the chamber of the main body, the electric toothbrush is positioned upside down such that a brush head of the electric toothbrush is closer to the bottom side of the main body than the top side of the main body.

4. The device of claim 1, wherein, when the electric toothbrush is inside the chamber of the main body, the electric toothbrush is positioned right side up such that a brush head of the electric toothbrush is closer to the top side of the main body than the bottom side of the main body.

5. The device of claim 1, wherein the bottom cover comprises vents.

6. The device of claim 1, wherein the upper cover comprises a rinsing cup when removed from the main body.

7. The device of claim 1, wherein the upper cover is transparent to visible light.

8. The device of claim 1, further comprising a switch disposed on an exterior surface of the main body and configured to indicate an operational status of the toothbrush sanitizing device.

9. The device of claim 1, wherein the charging coil is disposed proximate a handle of the electric toothbrush when the electric toothbrush is disposed in the chamber of the main body.

10. The device of claim 1, wherein the charging coil is disposed proximate a base of the electric toothbrush when the electric toothbrush is disposed in the chamber of the main body.

11. The device of claim 1, wherein the induction sensor is a Hall sensor.

12. The device of claim 1, further comprising a printed circuit board (PCB) disposed proximate a brush head of the electric toothbrush.

13. The device of claim 12, wherein the one or more ultraviolet lights are disposed on the PCB.

14. The device of claim 12, wherein the heater comprises a heating resistor disposed on the PCB.

15. A toothbrush sanitizing device for an electric toothbrush, the device comprising:
a main body having a chamber to receive the electric toothbrush,
a bottom cover removably attached to a bottom side of the main body,
an upper cover removably attached to a top side of the main body,
one or more ultraviolet lights configured to at least partially clean the electric toothbrush,
a heater configured to remove at least some moisture from the electric toothbrush, and
a magnetic coupler disposed on an interior surface of the main body and configured to removably couple the electric toothbrush to the toothbrush sanitizing device.

16. The device of claim 15, further comprising a charging coil disposed on the interior surface of the main body and configured to inductively charge the electric toothbrush.

17. The device of claim 15, further comprising a sensor configured to identify a presence of the electric toothbrush in the toothbrush sanitizing device.

18. The device of claim 15, wherein, when the electric toothbrush is inside the chamber of the main body, the electric toothbrush is positioned upside down such that a brush head of the electric toothbrush is closer to the bottom cover than the upper cover.

19. The device of claim 15, wherein, when the electric toothbrush is inside the chamber of the main body, the electric toothbrush is positioned right side up such that a brush head of the electric toothbrush is closer to the top cover than the bottom cover.

20. A method of storing and sanitizing an electric toothbrush, the method comprising:
inserting the electric toothbrush into a chamber of a main body of a toothbrush sanitizing device,
removably attaching an upper cover to a top side of the main body of the toothbrush sanitizing device,
detecting a presence of the electric toothbrush inside the main body of the toothbrush sanitizing device with a sensor, and
activating a plurality of functions of the toothbrush sanitizing device, the plurality of functions comprising:
inductively charging the electric toothbrush with a charging coil disposed on an interior surface of the main body of the toothbrush sanitizing device,
at least partially sanitizing the electric toothbrush with one or more ultraviolet lights, and
removing at least some moisture from the electric toothbrush with a heater.

21. The method of claim 20, wherein inserting the electric toothbrush into the chamber of the main body of the toothbrush sanitizing device comprises inserting the electric toothbrush upside down such that a brush head of the electric toothbrush is closer to the bottom side of the main body of the toothbrush sanitizing device than the top side of the main body of the toothbrush sanitizing device.

22. The method of claim 20, wherein inserting the electric toothbrush into the chamber of the main body of the toothbrush sanitizing device comprises inserting the electric toothbrush right side up such that a brush head of the electric toothbrush is closer to the top side of the main body of the toothbrush sanitizing device than the bottom side of the main body of the toothbrush sanitizing device.

23. The method of claim 20, further comprising removably coupling the electric toothbrush to the toothbrush sanitizing device with a magnetic coupler disposed on the interior surface of the main body.

24. The method of claim 20, further comprising removing a bottom cover to at least partially clean a bottom side of the main body of the toothbrush sanitizing device.

25. A toothbrush comprising,
a brush head;
a handle having a bottom side and a top side, wherein the brush head is connected to the handle at the top side of the handle,
a charging unit disposed inside the handle, the charging unit having a charging circuitry, the charging circuitry configured to inductively gain power from a charging coil of a toothbrush sanitizing device and charge a battery of the toothbrush,
a magnet disposed in the handle and configured to couple to a coupler on the toothbrush sanitizing device, and
a status indicator disposed on the handle and positioned closer to the bottom side than the top side of the handle.

26. The toothbrush of claim 25, further comprising a portable charging box configured to magnetically attach to the handle of the toothbrush via the magnet and provide power to the toothbrush.

27. A toothbrush comprising,
a brush head;
a handle having a bottom side and a top side, wherein the brush head is connected to the handle at the top side of the handle,
a charging unit on the handle, the charging unit having charging circuitry, the charging circuitry configured to inductively gain power from a charging coil of a toothbrush sanitizing device and charge a battery of the toothbrush,
a magnet disposed in the handle and configured to couple to a coupler on the toothbrush sanitizing device.

28. The toothbrush of claim 27, wherein the charging unit is disposed proximate the bottom side of the handle.

29. The toothbrush of claim 27, wherein the charging circuitry comprises a charging coil.

30. The toothbrush of claim 27, further comprising a status indicator disposed on the handle.

31. A charger for a toothbrush, the charger comprising,
a body having a surface sized and shaped to conform to a portion of an exterior surface of the toothbrush,
a charging circuitry inside the body configured to charge a battery of the toothbrush,
a magnet in the body configured to removably attach the body to the toothbrush.

32. The charger of claim 31, wherein the charger is configured to be secured to a support separate from the toothbrush.

33. The charger of claim 32, wherein the support comprises at least one of a wall, a counter, and a mirror.
